SEQUENCE LISTING (24 sequences)

SEQ ID NO: 1 - DNA, 24 bp, Artificial sequence
  The sequence is synthesized
tcctgagggtcttctctgtggagg

SEQ ID NO: 2 - DNA, 24 bp, Artificial Sequence
  The sequence is synthesized
tgtgcctggaatacatctccgaga

SEQ ID NO: 3 - DNA, 29 bp, Artificial sequence
  The sequence is synthesized.
taagacagatcaccttccaagagagacac

SEQ ID NO: 4 - DNA, 23 bp, Artificial sequence
  The sequence is synthesized
cacaggctgcactttagaggcaa

SEQ ID NO: 5 - DNA, 25 bp, Artificial Sequence
  The sequence is synthesized.
caacagcatgtgcttcatagttgcc

SEQ ID NO: 6 - DNA, 27 bp, Artificial sequence
  The sequence is synthesized
cgacagcactgcccactctagtaatag

SEQ ID NO: 7 - DNA, 30 bp, Artificial sequence
  The sequence is synthesized.
aacaaacacttgaagacactgaagaacaac

SEQ ID NO: 8 - DNA, 28 bp, Artificial sequence
  The sequence is synthesized.
tgctctcaactgaaaatggctatatgtc

SEQ ID NO: 9 - DNA, 23 bp, Artificial sequence
  The sequence is synthesized.
tcttccagggcaccttactgtcc

SEQ ID NO: 10 - DNA, 20 bp, Artificial sequence
  The sequence is synthesized.
accagcaaccccattccgag

SEQ ID NO: 11 - DNA, 23 bp, Artificial sequence
  The sequence is synthesized.
ttgatgtgtcccctgtgccttta

SEQ ID NO: 12 - DNA, 29 bp, Artificial sequence
  The sequence is synthesized.
acaagtttttgcctctagatgacactgtt

SEQ ID NO: 13 - DNA, 26 bp, Artificial sequence
  The sequence is synthesized.
aacccgaggaagtctaaatgaataat

SEQ ID NO: 14 - DNA, 26 bp, Artificial sequence
  The sequence is synthesized.
cacacccagttacccctgttattaac

SEQ ID NO: 15 - DNA, 27 bp, Artificial sequence.
  The sequence is synthesized.
tcctctctcatctgtagtctggcttta

SEQ ID NO: 16 - DNA, 26 bp, Artificial sequence
  The sequence is synthesized.
aaactataatagcaatctgtgcccaa

SEQ ID NO: 17 - DNA, 20 bp, Artificial sequence
  The sequence is synthesized.
agcattggtgcgtgtggtgc

SEQ ID NO: 18 - DNA, 25 bp, Artificial sequence
  The sequence is synthesized.
cctcttggtggaatctaggatcagg

SEQ ID NO: 19 - DNA, 26 bp, Artificial sequence
  The sequence is synthesized.
ttcaagtgaagttacctaatgctccc

SEQ ID NO: 20 - DNA, 26 bp, Artificial sequence
  The sequence is synthesized.
cctggggtacagaaatacttagtgat

SEQ ID NO: 21 - DNA, 26 bp, Artificial sequence
  The sequence is synthesized.
ttggaaagtctggatgggaatctttt

SEQ ID NO: 22 - DNA, 25 bp, Artificial sequence
  The sequence is synthesized.
aggggatttaacctacctttgtttc

SEQ ID NO: 23 - DNA, 26 bp, Artificial sequence
  The sequence is synthesized.
atgacaattaaattatcacgcttcca

SEQ ID NO: 24 - DNA, 26 bp, Artificial sequence
  The sequence is synthesized.
ttcttcttgtcagcagccacttatca

(19) United States
(12) Patent Application Publication
(10) Pub. No.: US 2008/0160545 A1
(43) Pub. Date: Jul. 3, 2008

(54) Pro-GRP as a surrogate marker to predict and monitor response to Bcl-2 inhibitor therapy

(75) Inventors: Evelyn M. McKeegan, Lake Forest, IL (US); Barry L. Dowell, Mundelein, IL (US); Rick R. Lesniewski, Pleasant Prairie, WI (US); Dimitri Semizarov, Chicago, IL (US)

Correspondence Address: VYSIS, INC;PATENT DEPARTMENT, 1300 E TOUHY AVENUE, DES PLAINES, IL, 60018, US

(73) Assignee: Abbott Laboratories, Abbott Park, IL (US)

(21) Appl. No.: 11/647,103

(22) Filed: Dec. 28, 2006

Related U.S. Application Data
(60) Provisional application No. 60/842,304.

(51) Int. Cl. G01N 33/574

(52) U.S. Cl. 435/7.23; 435/7.93

(57) ABSTRACT

A method for classifying cancer patients as eligible to receive cancer therapy with a Bcl-2 inhibitor comprising determination of the presence or absence in a patient tissue sample of levels of pro-GRP, as a surrogate marker for the presence of chromosomal copy number gain at chromosomal locus 18q21-q22. The classification of cancer patients based upon pro-GRP levels as a surrogate for the presence or absence of 18q21-q22 gain allows selection of patients to receive chemotherapy with a Bcl-2 family inhibitor, either as monotherapy or as part of combination therapy, and to monitor patient response to such therapy using a peripheral blood sample.

### REFERENCE TO RELATED APPLICATION

This application is a continuation-in-part application of U.S. Ser. No. 60/842,304, D. Semizarov et al., “Companion Diagnostic Assays for Cancer Therapy”, filed Sep. 5, 2006.

### FIELD OF THE INVENTION

This invention relates to diagnostic assays useful in classification of patients for selection of cancer therapy, and in particular relates to measurement of pro-GRP protein levels as a surrogate marker to identify patients eligible to receive Bcl-2-family antagonist therapy, either as monotherapy or as part of combination therapy, and that permit monitoring of patient response to such therapy.

### BACKGROUND OF THE INVENTION

Genetic heterogeneity of cancer is a factor complicating the development of efficacious cancer drugs. Cancers that are considered to be a single disease entity according to classical histopathological classification often reveal multiple genomic subtypes when subjected to molecular profiling. In some cases, molecular classification proved to be more accurate than the classical pathology. The efficacy of targeted cancer drugs may correlate with the presence of a genomic feature, such as a gene amplification, Cobleigh, M. A., et al., “Multinational study of the efficacy and safety of humanized anti-HER2 monoclonal antibody in women who have HER2-overexpressing metastatic breast cancer that has progressed after chemotherapy for metastatic disease”, J. Clin. Oncol., 17: 2639-2648, 1999; or a mutation, Lynch, T. J., et al., “Activating mutations in the epidermal growth factor receptor underlying responsiveness of non-small-cell lung cancer to gefitinib”, N. Engl. J. Med., 350: 2129-2139, 2004. For Her-2 in breast cancer, it has been demonstrated that detection of gene amplification provides superior prognostic and treatment selection information as compared with the detection by immunohistochemistry (IHC) of the protein overexpression, Pauletti, G., et al., “Assessment of Methods for Tissue-Based Detection of the HER-2/neu Alteration in Human Breast Cancer: A Direct Comparison of Fluorescence In Situ Hybridization and Immunohistochemistry”, J. Clin. Oncol., 18: 3651-3664, 2000. A need therefore exists for genomic classification markers that may improve the response rate of patients to targeted cancer therapy.

Lung cancer is an area of active research for new targeted cancer therapies. Lung malignancies are the leading cause of cancer mortality, which will result in approximately 160,000 deaths in the United States in 2006. Small-cell lung carcinoma (SCLC) is a histopathological subtype of lung cancer, which represents approximately 20% of lung cancer cases. The survival rate for this subtype is low (long-term survival 4-5%) and has not improved significantly in the past decade, despite the introduction of new chemotherapy regimens. The remainder of lung cancer cases are non-small-cell lung carcinomas (NSCLC), a category which is comprised of several common subtypes. In the past several years, there has been substantial progress in the development of targeted therapies for NSCLC, such as erlotinib and gefitinib. Genomic biomarkers have been discovered which enable stratification of NSCLC patients into potential responders and non-responders. In particular, mutations and amplifications in the EGFR kinase domain were shown to correlate with the response to erlotinib and gefitinib. Unfortunately, no such progress has been achieved with SCLC, even though genomic analysis of SCLC cell lines and tumors is reported in Ashman, J. N., et al., Chromosomal alterations in small cell lung cancer revealed by multicolour fluorescence in situ hybridization. Int. J. Cancer, 102: 230-236, 2002; 17; Coe, B. P., et al., “Gain of a region on 7p22.3, containing MAD1L1, is the most frequent event in small-cell lung cancer cell lines”, Genes Chromosomes Cancer, 45: 11-19, 2006; and Kim, Y. H., et al., “Combined microarray analysis of small cell lung cancer reveals altered apoptotic balance and distinct expression signatures of MYC family gene amplification”, Oncogene, 25: 130-138, 2006.

Targeted cancer therapy research has been reported against members of the Bcl-2 protein family, which are central regulators of programmed cell death. The Bcl-2 family members that inhibit apoptosis are overexpressed in cancers and contribute to tumorigenesis. Bcl-2 expression has been strongly correlated with resistance to cancer therapy and decreased survival. For example, the emergence of androgen independence in prostate cancer is characterized by a high incidence of Bcl-2 expression (≧40% of the cohort examined), see Chaudhary, K. S., et al., “Role of the Bcl-2 gene family in prostate cancer progression and its implications for therapeutic intervention” [Review], Environmental Health Perspectives 1999, 107, 49-57, which also corresponds to an increased resistance to therapy. Furthermore, overexpression of Bcl-2 in both NSCLC and SCLC cell lines, has been demonstrated to induce resistance to cytotoxic agents, Ohmori, T., et al., “Apoptosis of lung cancer cells caused by some anti-cancer agents (MMC, CPT-11, ADM) is inhibited by bcl-2”, Biochem. Biophys. Res. Commun. 1993, 192, 30-36. Yasui, K., et al., “Alteration in Copy Numbers of Genes as a Mechanism for Acquired Drug Resistance”, Can. Res. 2004, 64, 1403-1410, reports analysis of the etopside resistant ovarian cancer cell line SKOV3/VP for chromosome copy number gain. Yasui et al. describe copy number gain at the Bcl-w (BCL2L2) locus and conclude that Bcl-w expression is “at least partially responsible for the chemoresistance” of SKOV3/VP, Ibid. at p. 1409. Yatsui does not disclose identification of Bcl-2 family copy number change in any other cancer cell line.

Martinez-Climent, J. et al., “Transformation of follicular lymphoma to diffuse large cell lymphoma is associated with a heterogeneous set of DNA copy number and gene expression alterations”, Blood, 2003 Apr. 15; 101 (8): 3109-3116, describe identification of a copy number change at 18q21, including the Bcl-2 locus, in the transformation of follicular lymphoma to large cell lymphoma. Monni, O. et al., “DNA copy number changes in diffuse large B-cell lymphoma—comparative genomic hybridization study”, Blood, 1996 Jun. 15; 87 (12):5269-78, report multiple copy number changes in diffuse large B-cell lymphoma. Galteland, E. et al., “Translocation t(14;18) and gain of chromosome 18/BCL2: effects on BCL2 expression and apoptosis in B-cell non-Hodgkin's lymphomas”, Leukemia, 2005 December; 19 (12):2313-23, report gain of the chromosome locus of Bcl-2 in B-cell non-Hodgkin's lymphomas. Nupponen, N. et al., “Genetic alterations in hormone-refractory recurrent prostate carcinomas”, Am. J. Pathol., 1998 July; 153 (1):141-8, describe low level copy number gain of Bcl-2 in four of 17 samples of recurrent prostate cancer. These reports do not correlate copy number gain at 18q21 with therapy resistance.

A compound called ABT-737 is a small-molecule inhibitor of the Bcl-2 family members Bcl-2, Bcl-XL, and Bcl-w, and has been shown to induce regression of solid tumors, Oltersdorf, T., “An inhibitor of Bcl-2 family proteins induces regression of solid tumours”, Nature, 435: 677-681, 2005. ABT-737 has been tested against a diverse panel of human cancer cell lines and has displayed selective potency against SCLC and lymphoma cell lines, Ibid. ABT-737's chemical structure is provided by Oltersdorf et al. at p. 679.

Progastrin releasing peptide (“pro-GRP”) has been identified as a specific marker of small cell lung cancer, see Y. Miyake et al., “Pro-gastrin-releasing peptide (31-98) is a specific tumor marker in patients with small cell lung cancer”, Cancer Research 1994, April 15; 54(8): 2136-40; and K. Aoyagi et al., “Enzyme Immunoassay of Immunoreactive Progastrin-Releasing Peptide (31-98) as Tumor Marker for Small-Cell Lung Carcinoma: Development and Evaluation”, Clinical Chemistry, 41(4): 537-543 (1995), incorporated herein by reference, cited hereafter as “Aoyagi et al.” ELISA based assays for pro-GRP levels in serum fractions from patient blood samples are in clinical use in Japan to diagnose small cell lung cancer and to monitor patient response to conventional chemotherapy for small cell lung cancer.

Because of the potential therapeutic use of inhibitors for Bcl-2 family members, companion diagnostic assays that would identify patients eligible to receive Bcl-2 family inhibitor therapy are needed. Additionally, there is a clear need to support this therapy with diagnostic assays using biomarkers that would facilitate monitoring the efficacy of Bcl-2 family inhibition therapy. There is a further need for companion assays using markers that can be measured in a readily obtainable sample such as blood or a blood plasma fraction.

### SUMMARY OF THE INVENTION

The invention provides companion diagnostic assays for classification of patients for cancer treatment which comprise assessment in a patient tissue sample of levels of pro-GRP as a surrogate marker of the presence of chromosomal copy number gain at the chromosome 18q21-q22 locus in the patient tumor. This chromosome locus includes the Bcl-2 gene and the pro-GRP gene at 18q21.3. The inventive assays include assay methods for identifying patients eligible to receive Bcl-2 antagonist therapy and for monitoring patient response to such therapy. The invention preferably comprises determining by immunoassay, levels of pro-GRP, of a pro-GRP precursor, or of fragments of either pro-GRP or a pro-GRP precursor in a blood plasma sample. Patients classified as having increased levels of pro-GRP (of a pro-GRP precursor, or of fragments of either pro-GRP or a pro-GRP precursor) are eligible to receive anti-Bcl-2 therapy because they are more likely to be respond to this therapy. Applicants believe that in patients whose tumors exhibit the 18q copy number gain, pro-GRP levels are increased also because pro-GRP maps close to Bcl-2 and the Bcl-2 locus amplification leads to pro-GRP upregulation. Thus, determination of the increased levels of pro-GRP can be used as a bcl-2 inhibitor therapy stratification marker.

In a preferred embodiment, the invention comprises a method for identifying a patient as eligible to receive Bcl-2 inhibitor therapy comprising: (a) providing a blood sample from a patient; (b) determining levels of pro-GRP in the blood sample; and (c) identifying the patient as eligible for Bcl-2 family inhibitor therapy where the patient's sample is classified as having increased levels of pro-GRP. In this embodiment, the pro-GRP level is preferably determined by an immunoassay performed on a blood serum or, more preferably on a plasma fraction of the blood sample.

The invention also comprises a method for monitoring a patient being treated with Bcl-2 inhibitor therapy comprising: (a) providing a peripheral blood sample from a patient; (b) measuring levels of pro-GRP, a pro-GRP precursor, or fragments of either pro-GRP or a pro-GRP precursor in the peripheral blood sample and (c) comparing the level pro-GRP in the peripheral blood sample relative to the patient baseline blood level of pro-GRP. Applicants expect that decreases in pro-GRP levels occurring over the course of therapy are indicative of therapeutic response to the Bcl-2 inhibitor. Applicants also expect that increases in pro-GRP levels may be seen in the period after the start of bcl-2 inhibitor therapy, and that these increases also mark positive response. These short term increases are expected because if the patient is responding, tumor cells will become apoptotic, die and be absorbed into the circulation, resulting in increases in pro-GRP levels originating in the dead tumor cells. Applicants further expect that over time when the therapy is effective, the temporary spike in pro-GRP levels will disappear, the number of tumor cells will decrease and pro-GRP levels in serum or plasma will decrease proportionately.

The invention further comprises a reagent kit for an assay for classification of a patient for cancer therapy, such as eligibility for Bcl-2 inhibitor therapy, or for monitoring response to such therapy, comprising a container comprising at least one labeled antibody or protein capable of specific binding to pro-GRP, a pro-GRP precursor, or fragments of either pro-GRP or a pro-GRP precursor. In a preferred embodiment, the reagent kits of the invention also comprise a pro-GRP calibration sample.

The invention has significant capability to provide improved stratification of patients for Bcl-2 inhibitor therapy. The assessment of pro-GRP levels with the invention also allows tracking of individual patient response to the therapy using a readily obtainable patient sample. The inventive assays have particular utility for treatment of SCLC and lymphoma patients with Bcl-2 inhibitors, for example ABT-737, ABT-263 or analogs thereof.

### BRIEF DESCRIPTION OF THE DRAWINGS

FIG. 1 shows a plot of experimental quantitative PCR determination of chromosomal copy number on chromosome arm 18q in various SCLC cell lines sensitive and resistant to ABT-737.

FIG. 2 depicts the relationship between the Bcl-2 gene copy number of SCLC cell lines and sensitivity of the cell lines to ABT-737.

FIG. 3 shows classification of a 62 patient cohort of clinical SCLC samples by chromosome copy number of the Bcl-2 locus.

### DETAILED DESCRIPTION OF THE INVENTION

I. General

The invention is based on the discovery by Applicants of chromosome copy number changes in small cell lung cancer cell lines that correlate to therapy sensitivity. In particular, Applicants correlated chromosome copy number gain at 18q21-q22 to sensitivity to a Bcl-2 family inhibitor. The Bcl-2 gene in this locus is a key regulator of cell survival, and other genes in this locus such as NOXA also impact cell survival. Chromosomal gain at 18q21-q22 can thus mark sensitivity to other cancer therapy, such as other chemotherapy or radiation therapy. Applicants noted that pro-GRP maps into the 18q amplified locus, close to Bcl-2, and determined that pro-GRP levels in patient tissue could thus be used as a surrogate marker of the presence of the amplied locus.

As used herein, a “Bcl-2 inhibitor” refers to a therapeutic compound of any type, including small molecule-, antibody-, antisense-, small interfering RNA-, or microRNA-based compounds, that binds to at Bcl-2, and antagonizes the activity of the Bcl-2 related nucleic acid or protein. The inventive methods are useful with any known or hereafter developed Bcl-2 inhibitor. One Bcl-2 inhibitor is ABT-737, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide, which binds to each of Bcl-2, Bcl-XL, and Bcl-w. Another Bcl-2 inhibitor is ABT-263, N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide. The chemical structure of ABT-263 is

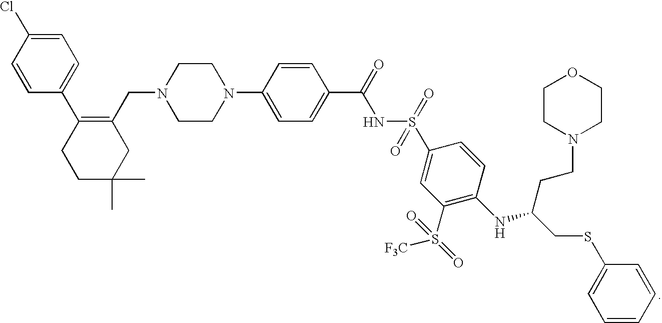

Use of the inventive pro-GRP assays for selection of patients eligible for therapy with analogs of either ABT-737 or ABT-263 are another embodiment of the invention.

The assays of the invention have potential use with targeted cancer therapy. In particular, the inventive assays are useful with therapy selection for small cell lung cancer and lymphoma patients, such as therapy with Bcl-2 inhibitors. The inventive assays are useful as companion assays for Bcl-2 inhibitor therapy, given either as monotherapy or as part of combination therapy with other chemotherapy, such as convention chemotherapy. The pro-GRP assays can be performed in relation to any cancer type in which copy number gain of Bcl-2 is involved. Other examples of such cancers include solid tissue epithelial cancers, e.g. prostate cancer, ovarian and esophageal cancer. The inventive assays are performed on a patient tissue sample of any type or on a derivative thereof, including peripheral blood, serum or plasma fraction from peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates.

As used herein, Bcl-2 (official symbol BCL2) means the human B-cell CLL/lymphoma 2 gene; Bcl-xl (official symbol BCL2L1) means the human BCL2-like 1 gene; Bcl-w (official symbol BCL2L2) means the human BCL2-like 2 gene; pro-GRP (official symbol GRP) means the human gastrin releasing peptide; NOXA (official symbol PMAIP1) means the human phorbol-12-myristate-13-acetate-induced protein 1 gene; ABL1 (official symbol ABL1) means the human Abelson murine leukemia viral oncogene homolog 1 gene; RAC1 (official symbol RAC1) means the human ras-related C3 botulinum toxin substrate 1 gene; RASSF3 (official symbol RASSF3) means the human Ras association (RalGDS/AF-6) domain family 3 gene; RAB22A (official symbol RAB22A) means the human member RAS oncogene family gene; BI-1 or BAX inhibitor 1 (official symbol TEGT) means the human testis enhanced gene transcript gene; FAIM-2 (official symbol FAIM) means the human Fas apoptotic inhibitory molecule gene; and RFC2 (official symbol RFC2) means the human replication factor C (activator 1) 2 gene. As used herein, the term “official symbol” refers to EntrezGene database maintained by the United States National Center for Biotechnology Information.

Chromosomal loci cited herein are based on Build 35 of the Human Genome Map, as accessed through the University of California Santa Cruz Genome Browser. As used herein, reference to a chromosome locus or band, such as 18q21, refers to all of the loci or sub bands, for example, such as 18q21.1 or 18q21.3, within the locus or the band.

As used herein, pro-GRP levels include any of levels of the expressed protein of pro-GRP, of the expressed protein of a pro-GRP precursor, or a fragment of either of the expressed protein of pro-GRP or of a pro-GRP precursor.

II. Bcl-2 Family Inhibitor Biomarkers

The invention was developed by assessment in a patient tissue sample of chromosome copy number change at chromosome locus 18q21-q22, preferably at either chromosome band 18q21-q22 or band 14q11, and more preferably at both 18q21-q22 and 14q11. Chromosome region 18q21-q22 encompasses the chromosomal DNA sequence of the Bcl-2 gene and the pro-GRP gene at 18q21.3 and the NOXA gene at 18q21.32. Chromosome region 14q11 encompasses the chromosomal DNA sequence of the Bcl-w gene at 14q11.2. It is also within the invention to assess the chromosomal locus of the Bcl-XL gene at 20q11.2. Applicants prefer, however, to assess the 18q21-q22 and 14q11 discriminant regions as gains of these loci were correlated to SCLC sensitivity to ABT-737, whereas gain of 20q11.2 showed no correlation to ABT-737 sensitivity.

These genomic biomarkers were identified by Applicants through comparative genomic hybridization (CGH) analysis of 23 SCLC cell lines used to test Bcl-2 inhibitors in vitro and in vivo and investigation of their clinical significance. These genomic biomarkers are of particular interest for use in companion diagnostic assays to the use of ABT-737 Bcl-2 inhibitor therapy against SCLC and lymphoma. Although Zhao, X., et al., “Homozygous deletions and chromosome amplifications in human lung carcinomas revealed by single nucleotide polymorphism array analysis”, Cancer Res., 65: 5561-5570, 2005 (hereafter referred to as Zhao et al.), reports on the genome-wide analysis of 5 SCLC cell lines and 19 SCLC patient tumors using 100K SNP genotyping microarrays, Zhao et al. do not disclose chromosome copy number gain at 18q21-q22 nor at 14q11.

Applicants' investigation further revealed multiple other novel regions of chromosome copy number change not previously reported in SCLC. These other novel genomic biomarkers are listed in Table 1 below and are also not reported in Zhao et al. A gain of the locus of ABL1 at 9q34 can be potentially used to identify patients for treatment with the ABL1 kinase inhibitor imatinib mesylate, Gleevec® (Gleevec is a registered trademark of Novartis). Copy number gains at three members of the Ras family, RAC1 at 7p22.1 (gains in 69% of lines and 66% of 19 tumors studied), RASSF3 at 12q24 (65% of lines and 70% of 19 tumors studied), and RAB22A at 20q13.3 (42% of lines and 84% of 19 tumors studied), are notable because of the known oncogenic impact of Ras family genes and the high percentage occurrence in the tumor cohort studied. Gains at other anti-apoptotic genes were seen for BI-1 at 12q12-q14, FAIM-2 (gained in 73% of lines and 58% of 19 tumors studied) at 12q13.12, and RFC2 (gained in 71% of lines and 60% of 19 tumors studied) at 7q11. Diagnostic assays for detecting any of these copy number changes in small cell lung cancer or other cancer is another embodiment of the invention.

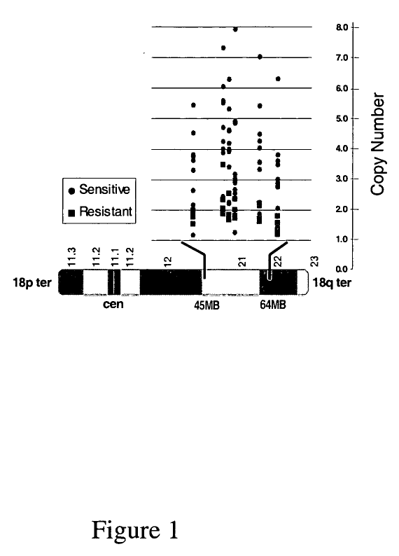

Applicants used a bioinformatics approach that identified regions of chromosomal aberrations that discriminate between cell line groups that were sensitive and resistant to ABT-737. This approach tested for statistical significance using Fisher's Exact Test to determine if a SNP identified through the CGH analysis shows preferential gain/loss in the sensitive or resistant group. The copy number thresholds for amplifications and deletions used in this analysis were set at 2.8 and 1.5, respectively. Contiguous regions of probesets (SNPs) with low table and two-sided p-values were then subjected to further analysis. One large region on chromosome 18q was of particular interest because of high copy numbers and low p-values. This region spans chromosomal bands 18q21.1 through 18q22. Applicants then used real-time qPCR to validate this region as a potential therapy stratification marker. qPCR was used to evaluate six loci starting at 48 Mb (18q21.1) and ending at 62 Mb (18q22) within chromosome 18. The qPCR results are displayed in FIG. 1 and show segregation between the sensitive and resistant lines based on the copy number of the test locus (ANOVA test p-value <0.0001). The sensitive lines carry an amplification of the region under consideration (3 to 7 copies), whereas the resistant lines display a normal copy number. The target of ABT-737, Bcl-2, is located within this discriminant region and had a low 0.04 p-value for significance in determining sensitivity. Applicants then analyzed a 62 patient SCLC cohort for copy number gains at 18q21-q22 and found copy number gain in 48% of this cohort, with low-level amplifications of the Bcl-2 gene present in 40% of the patients (25 out of 62) and high-level amplifications in 8% of the tumors (5 out of 62).

Assessment of copy number gain at the 18q21-q22 and 14q11 discriminant regions are believed applicable for patient classification for other cancer chemotherapy, such as treatment with cytotoxic drugs, DNA-damaging drugs, tubulin inhibitors, tyrosine kinase inhibitors, and anti-metabolites. The Bcl-2 genes provide significant cell survival benefit, and their chromosome copy number gain driving their expression is expected to mark therapy resistance.

III. Assays

Nucleic acid assay methods for detection of chromosomal DNA copy number changes include: (i) in situ hybridization assays to intact tissue or cellular samples, (ii) microarray hybridization assays to chromosomal DNA extracted from a tissue sample, and (iii) polymerase chain reaction (PCR) or other amplification assays to chromosomal DNA extracted from a tissue sample. Assays using synthetic analogs of nucleic acids, such as peptide nucleic acids, in any of these formats can also be used.

The assays of the invention are used to identify the pro-GRP surrogate biomarker for both predicting therapy response and for monitoring patient response to Bcl-2 inhibitor therapy. Assays for response prediction are run before start of therapy and patients showing pro-GRP increases marking the presence of chromosome copy number gains are eligible to receive Bcl-2 inhibitor therapy. For monitoring patient response, the assay is run at the initiation of therapy to establish baseline levels of the pro-GRP biomarker in the tissue sample, for example, the percent of total cells or number of cells showing the copy number gain in the sample. The same tissue is then sampled and assayed and the levels of the biomarker compared to the baseline. Where the levels remain the same or decrease, the therapy is likely being effective and can be continued. Where significant increase over baseline level occurs, the patient may not be responding. Preferably, the baseline level is determined in a peripheral blood sample taken from the patient at the time of start of therapy.

Detection of the genomic biomarkers is done by hybridization assays using detectably labeled nucleic acid-based probes, such as deoxyribonucleic acid (DNA) probes or protein nucleic acid (PNA) probes, or unlabeled primers which are designed/selected to hybridize to the specific designed chromosomal target. The unlabeled primers are used in amplification assays, such as by polymerase chain reaction (PCR), in which after primer binding, a polymerase amplifies the target nucleic acid sequence for subsequent detection. The detection probes used in PCR or other amplification assays are preferably fluorescent, and still more preferably, detection probes useful in “real-time PCR”. Fluorescent labels are also preferred for use in situ hybridization but other detectable labels commonly used in hybridization techniques, e.g., enzymatic, chromogenic and isotopic labels, can also be used. Useful probe labeling techniques are described in Molecular Cytogenetics: Protocols and Applications, Y.-S. Fan, Ed., Chap. 2, “Labeling Fluorescence In Situ Hybridization Probes for Genomic Targets”, L. Morrison et. al., p. 21-40, Humana Press, ©2002, incorporated herein by reference. In detection of the genomic biomarkers by microarray analysis, these probe labeling techniques are applied to label a chromosomal DNA extract from a patient sample, which is then hybridized to the microarray.

Iin situ hybrization is used to detect the presence of chromosomal copy number increase or gene amplification at either or both of the 18q21-q22 or 14q11 loci, or at the other novel genomic biomarker regions. Probes for use in the in situ hybridization methods of the invention fall into two broad groups: chromosome enumeration probes, i.e., probes that hybridize to a chromosomal region, usually a repeat sequence region, and indicate the presence or absence of an entire chromosome, and locus specific probes, i.e., probes that hybridize to a specific locus on a chromosome and detect the presence or absence of a specific locus. It is preferred to use a locus specific probe that can detect changes of the unique chromosomal DNA sequences at the interrogated locus such as 18q21-q22. Methods for use of unique sequence probes for in situ hybridization are described in U.S. Pat. No. 5,447,841, incorporated herein by reference.

A chromosome enumeration probe can hybridize to a repetitive sequence, located either near or removed from a centromere, or can hybridize to a unique sequence located at any position on a chromosome. For example, a chromosome enumeration probe can hybridize with repetitive DNA associated with the centromere of a chromosome. Centromeres of primate chromosomes contain a complex family of long tandem repeats of DNA comprised of a monomer repeat length of about 171 base pairs, that are referred to as alpha-satellite DNA. Centromere fluorescence in situ hybridization probes to each of chromosomes 14 and 18 are commercially available from Abbott Molecular (Des Plaines, Ill.).

In situ hybridization probes employ directly labeled fluorescent probes, such as described in U.S. Pat. No. 5,491,224, incorporated herein by reference. U.S. Pat. No. 5,491,224 also describes simultaneous FISH assays using more than one fluorescently labeled probe. Use of a pair of fluorescent probes, for example, one for the 18q21-q22 locus of Bcl-2 and one for the centromere of chromosome 18, or one for the 14q11 locus of Bcl-w and one for the centromere of chromosome 14, allows determination of the ratio of the gene locus copy number to the centromere copy number. This multiplex assay can provide a more precise identification of copy number increase through determination on a cell-by-cell basis of whether gene amplification, ie. a ratio of the number of the gene locus probe signals to the centromere probe signals in each cell that is greater than 2, exists, or whether gain of the entire chromosome has occurred, ie. a ratio of the number of the gene locus probe signals to the centromere probe signals in each cell of 1/1 to less than 2/1, but with more than the normal number of two gene locus probe signals. Samples that are classified as amplified from dual probe analysis with ratios of 2/1 or greater, or those having three or more gene locus probe signals, either in dual probe or single probe analysis, are identified as having the chromosomal gain related to Bcl-2 inhibitor therapy.

Useful locus specific probes can be produced in any manner and will generally contain sequences to hybridize to a chromosomal DNA target sequence of about 10,000 to about 1,000,000 bases long. Preferably the probe will hybridize to a target stretch of chromosomal DNA at the target locus of at least 100,000 bases long to about 500,000 bases long, and will also include unlabeled blocking nucleic acid in the probe mix, as disclosed in U.S. Pat. No. 5,756,696, herein incorporated by reference, to avoid non-specific binding of the probe. It is also possible to use unlabeled, synthesized oligomeric nucleic acid or peptide nucleic acid as the blocking nucleic acid or as the centromeric probe. For targeting the particular gene locus, it is preferred that the probes include nucleic acid sequences that span the gene and thus hybridize to both sides of the entire genomic coding locus of the gene. The probes can be produced starting with human DNA containing clones such as Bacterial Artificial Chromosomes (BAC's) or the like. BAC libraries for the human genome are available from Invitrogen and can be investigated for identification of useful clones. It is preferred to use the University of California Santa Cruz Genome Browser to identify DNA sequences in the target locus. These DNA sequences can then be used to identify useful clones contained in commercially available or academic libraries. The clones can then be labeled by conventional nick translation methods and tested as in situ hybridization probes.

Examples of fluorophores that can be used in the in situ hybridization methods described herein are: 7-amino-4-methylcoumarin-3-acetic acid (AMCA), Texas Red™ (Molecular Probes, Inc., Eugene, Oreg.); 5-(and-6)-carboxy-X-rhodamine, lissamine rhodamine B, 5-(and-6)-carboxyfluorescein; fluorescein-5-isothiocyanate (FITC); 7-diethylaminocoumarin-3-carboxylic acid, tetramethyl-rhodamine-5-(and-6)-isothiocyanate; 5-(and-6)-carboxytetramethylrhodamine; 7-hydroxy-coumarin-3-carboxylic acid; 6-[fluorescein 5-(and-6)-carboxamido]hexanoic acid; N-(4,4-difluoro-5,7-dimethyl-4-bora-3a,4a diaza-3-indacenepropionic acid; eosin-5-isothiocyanate; erythrosine-5-isothiocyanate; 5-(and-6)-carboxyrhodamine 6G; and Cascade™ blue aectylazide (Molecular Probes).

Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems, such as those available from MetaSystems or Applied Imaging. Alternatively, techniques such as flow cytometry can be used to examine the hybridization pattern of the chromosomal probes.

Although a cell-by-cell copy number analysis results from in situ hybridization, the genomic biomarkers can also be determined by quantitative PCR. In this method, chromosomal DNA is extracted from the tissue sample, and is then amplified by PCR using a pair of primers specific to at least one of Bcl-2, Bcl-xl or Bcl-w, or by multiplex PCR, using multiple pairs of primers. Any primer sequence for the biomarkers can be used. The copy number of the tissue is then determined by comparison to a reference amplification standard, Microarray copy number analysis can also be used. The chromosomal DNA after extraction is labeled for hybridization to a microarray comprising a substrate having multiple immobilized unlabeled nucleic acid probes arrayed at probe densities up to several million probes per square centimeter of substrate surface. Multiple microarray formats exist and any of these can be used, including microarrays based on BAC's and on oligonucleotides, such as those available from Agilent Technologies (Palo Alto, Calif.), and Affymetrix (Santa Clara, Calif.). When using a oligonucleotide microarray to detect chromosomal copy number change, it is preferred to use a microarray that has probe sequences to more than three separate locations in the targeted region.

IV. Immunoassays and Protein Assays

Protein assay methods useful in the invention to measure pro-GRP levels comprise (i) immunoassay methods involving binding of a labeled antibody or protein to the expressed protein of pro-GRP, a pro-GRP precursor or a fragment thereof, (ii) mass spectrometry methods to determine expressed protein of pro-GRP, a pro-GRP precursor or fragment thereof, and (iii) proteomic based or “protein chip” assays for the expressed protein of pro-GRP, a pro-GRP precursor or fragment thereof. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

A preferred immunoassay is sandwich type format, wherein antibodies are employed to separate and quantify pro-GRP levels in the test sample or test sample extract. More specifically, at least two antibodies bind to different parts of the pro-GRP, pro-GRP precursor or fragment thereof, forming an immune complex which is referred to as a “sandwich”. Generally, one or more antibodies can be used to capture the pro-GRP target in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, it is preferred that both antibodies binding to the target are not diminished by the binding of any other antibody in the assay to its respective binding site. In other words, antibodies should be selected so that the one or more first antibodies brought into contact with a test sample or test sample extract do not bind to all or part of the binding site recognized by the second or subsequent antibodies, thereby interfering with the ability of the one or more second detection antibodies to bind. In a sandwich assay, the antibodies, preferably, the at least one capture antibody, are used in molar excess amounts of the maximum amount of pro-GRP expected in the test sample or test sample extract. For example, from about 5 μg/mL to about 1 mg/mL of antibody per mL of solid phase containing solution can be used.

As used herein, an “antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology.

Thus, the term “antibody,” as used herein also includes antibody fragments either produced by the modification of whole antibodies or synthesized de novo using recombinant DNA methodologies. Useful antibodies include single chain antibodies (antibodies that exist as a single polypeptide chain), more preferably single chain Fv antibodies (sFv or scFv), in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, light and heavy polypeptide chains from an antibody V region into a molecule that folds into a three dimensional structure substantially similar to the structure of an antigen-binding site are known to those of skill in the art (see, e.g., U.S. Pat. Nos. 5,091,513, 5,132,405, and 4,956,778).

Any suitable antibodies or binding proteins that bind to pro-GRP, a pro-gRP precursor or a fragment thereof can be used. Monoclonal antibodies are preferred, and suitable ELISA assay kits for pro-GRP are available from IBL (Hamburg, Germany). Mononclonal antibodies for binding to a pro-GRP precursor and suitable for use herein are disclosed in U.S. Pat. No. 5,550,026, K. Yamaguchi et al., “Antibodies To Human Gastrin-Releasing Peptide Precursor and Use Thereof”, incorporated herein by reference. Suitable antibodies are also disclosed in Aoyagi et al. The biomarker-antibody/protein immune complexes formed in these assays can be detected using any suitable technique. Any suitable label can be used. The selection of a particular label is not critical, but the chosen label must be capable of producing a detectable signal either by itself or in conjunction with one or more additional substances.

Useful detectable labels, their attachment to antibodies and detection techniques therefore are known in the art. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline peroxidase, glucose 6-phosphate dehydrogenase, etc., a chemiluminescent label, such as, acridinium derivatives, luminol, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y.(1997) and in Haugland, Handbook of Fluorescent Probes and Research Chemi (1996), which is a combined handbook and catalogue published by Molecular Probes, Inc., Eugene, Oreg., each of which is incorporated herein by reference. Preferred labels for use with the invention are chemiluminscent labels such as acridinium-9-carboxamide. Additional detail can be found in Mattingly, P. G., and Adamczyk, M. (2002) Chemiluminescent N-sulfonylacridinium-9-carboxamides and their application in clinical assays, in Luminescence Biotechnology: Instruments and Applications (Dyke, K. V., Ed.) pp 77-105, CRC Press, Boca Raton.

The detectable label can be bound to the analyte or antibody either directly or through a coupling agent. An example of a coupling agent that can be used is EDAC (1-ethyl-3-(3-dimethylaminopropyl) carbodiimide, hydrochloride) that is commercially available from Sigma-Aldrich (St. Louis, Mo.). Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The capture antibody can be bound to a solid support which facilitates the separation of the antibody-pro-GRP complex from the test sample. The type of solid support or “solid phase” used in the inventive immunoassay is not critical and can be selected by one skilled in the art. A solid phase or solid support, as used herein, refers to any material that is insoluble, or can be made insoluble by a subsequent reaction. Useful solid phases or solid supports are known to those in the art and include the walls of wells of a reaction tray, test tubes, polystyrene beads, magnetic beads, nitrocellulose strips, membranes, microparticles such as latex particles, sheep (or other animal) red blood cells, and Duracytes® (a registered trademark of Abbott Laboratories, Abbott Park, Ill.), which are red blood cells “fixed” by pyruvic aldehyde and formaldehyde, and others. Suitable methods for immobilizing peptides on solid phases include ionic, hydrophobic, covalent interactions and the like. The solid phase can be chosen for its intrinsic ability to attract and immobilize the capture reagent. Alternatively, the solid phase can comprise an additional receptor which has the ability to attract and immobilize the capture reagent. The additional receptor can include a charged substance that is oppositely charged with respect to the capture reagent itself or to a charged substance conjugated to the capture reagent.

After the test sample or test sample extract is brought into contact with the at capture antibody, the resulting mixture is incubated to allow for the formation of a first capture antibody—pro-GRP complex. The incubation can be carried out at any suitable pH, including a pH of from about 4.5 to about 10.0, at any suitable temperature, including from about 2° C. to about 45° C., and for a suitable time period from at least about one (1) minute to about eighteen (18) hours, and preferably from about 4-20 minutes.

After formation of the labeled complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. For solution phase immunoassays, once the amount of the label in the complex has been quantified, the concentration of biomarker in the test sample is determined by use of a standard curve that has been generated using serial dilutions of the biomarker of known concentration. Other than using serial dilutions of the biomarker, the standard curve can be generated gravimetrically, by mass spectroscopy and by other techniques known in the art.

For IHC assays for pro-GRP, detection of the antibody-antigen binding is preferably done using a conjugated enzyme label attached to a secondary binding antibody, such as horseradish perioxidase. These enzymes in the presence of colored substrate, produce at the site of the binding a colored deposit, called the stain, which can be identified under a light microscope. The site and extent of the staining is then identified and classifed. In addition to manual inspection of the slide, automated IHC imaging techniques are known to the art and can be used.

V. Sample Processing and Assay Performance

The tissue sample to be assayed by the inventive methods can comprise any type, including a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or plasma fraction of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, a plasma fraction or a serum fraction, and this extract, plasma fraction or serum fraction can then be assayed. A microdissection of the tissue sample to obtain a cellular sample enriched with suspected tumor cells can also be used. The preferred tissue samples for use herein are peripheral blood and serum fractions thereof.

The tissue sample can be processed by any desirable method for performing protein-based assays. For in situ hybridization assays potentially used with the inventive assays to confirm the presence of the Bcl-2 18q copy number gain, a paraffin embedded tumor tissue sample or bone marrow sample is fixed on a glass microscope slide and deparaffinized with a solvent, typically xylene. Useful protocols for tissue deparaffinization and in situ hybridization are available from Abbott Molecular Inc. (Des Plaines, Ill.). Any suitable instrumentation or automation can be used in the performance of the inventive assays. PCR based assays can be performed on the m2000 instrument system (Abbott Molecular, Des Plaines, Ill.). Automated imaging can be employed for the preferred fluorescence in situ hybridization assays.

In another confirmatory assay for the presence of chromosomal copy number gain, the sample comprises a peripheral blood sample from a patient which is processed to produce an extract of circulating tumor cells having increased chromosomal copy number of at least one of 18q21-q22 and 14q 1.2. The circulating tumor cells can be separated by immunomagnetic separation technology such as that available from Immunicon (Huntingdon Valley, Pa.). The number of circulating tumor cells showing at least one copy number gain is then compared to the baseline level of circulating tumor cells having increased copy number determined preferably at the start of therapy. Increases in the number of such circulating tumor cells can indicate therapy failure.

Test samples for assays to confirm copy number gain presence can comprise any number of cells that is sufficient for a clinical diagnosis, and typically contain at least about 100 cells. In a typical FISH assay, the hybridization pattern is assessed in about 25-1,000 cells. Test samples are typically considered “test positive” when found to contain the chromosomal gain in a sufficient proportion of the sample. The number of cells identified with chromosomal copy number and used to classify a particular sample as positive, in general will vary with the number of cells in the sample. The number of cells used for a positive classification is also known as the cut-off value. Examples of cutoff values that can be used in the determinations include about 5, 25, 50, 100 and 250 cells, or 5%, 10%, 15%, 20%, 25%, 30%, 35%, 40%, 50% and 60% of cells in the sample population. As low as one cell may be sufficient to classify a sample as positive. In a typical paraffin embedded tissue sample, it is preferred to identify at least 30 cells as positive and more preferred to identify at least 20 cells as positive for having the chromosomal copy number gain. For example, detection in a typical paraffin embedded small cell lung cancer tissue of 30 cells having gain of 18q21-q22 would be sufficient to classify the tissue as positive and eligible for treatment with ABT-737.

For the preferred immunoassays to a peripheral blood sample, it is preferred to start with a conventional 10 milliliter peripheral blood sample from the patient. The sample may be pretreated, as necessary or desired, by dilution in an appropriate buffer solution or other solution, or optionally may be concentrated. Any of a number of standard aqueous buffer solutions, employing any of a variety of buffers, such as phosphate, Tris, or the like, optionally at physiological pH, can be used. Additives for improving stability of pro-GRP in the patient sample can be used. For example, known protease inhibitors such as PMSF and EDTA can be used to prevent or delay proteolytic cleavage of pro-GRP that can occur during storage of the blood sample before processing. The sample is then processed by any suitable technique to produce a blood plasma or serum fraction. The blood plasma or serum fraction is then used in an immunoassay to determine the pro-GRP levels.

The preferred immunoassays can also be performed manually or on any suitable automated immunoassay apparatus, including the Architect® or AxSym® systems (a registered trademark of Abbott Diagnostics, Abbott Park, Ill.), the Centaur® system (a registered trademark of Bayer Diagnostics, Tarreytown, N.Y.), the UniCel® DxC 6001 Synchron Access Clinical System (a registered trademark of Beckman Coulter, Fullerton, Calif.), the Dimension® RxL Max System (a registered trademark of Dade-Behring, Deerfield, Ill.) and the Elecsys 2010 system (Roche Diagnostics, Indianapolis, Ind.). The Architect instrument carries out automatically the steps of incubating the test sample or test sample extract with a capture antibody, washing the resulting analyte-capture antibody complex, adding an acridinium labeled second antibody that binds to the analyte, incubating the mixture of the labeled second antibody and analyte-capture antibody complex, washing the resulting complex, adding a signal generating solution to the mixture that triggers the chemiluminescent acridinium label, measuring the amount of chemiluminescence and determining the amount of analyte present. The Architect determines the amount of analyte present by signal measurements of the emitted chemiluminescence in RLUs (Relative Light Units), which are the designation for the optical unit of measurement utilized on the ARCHITECT systems. The ARCHITCT optics system is essentially a photomultiplier tube (PMT) that performs photon counting on the light emitted by the chemiluminescent reaction. The amount of light generated by the chemiluminescent reaction is proportional to the amount of acridinium tracer present in the reaction mixture, and thereby allows quantitation of the patient sample analyte that is also proportional to the amount of acridinium remaining in the reaction mixture at the time the chemiluminescent reaction occurs.

VI. Assay Kits

In another aspect, the invention comprises kits for the measurement of pro-GRP levels that comprise containers containing at least one labeled protein or antibody specific for binding to at least one of the expressed protein of pro-GRP, a pro-GRP precursor or fragments thereof. These kits may also include containers with other associated reagents for the assay. Preferred kits of the invention comprise containers containing, a labeled monoclonal antibody for binding to pro-GRP, a pro-GRP precursor or a fragment thereof and at least one calibrator composition.

### VII. EXPERIMENTAL

### Example 1

The following Example 1 describes Applicants' performance of a series of experiments. First, a whole-genome screen with high-density SNP genotyping arrays identified recurrent gene amplifications/deletions in SCLC cells. Novel recurrent chromosomal copy number gains were identified, were confirmed by real-time qPCR, and were then validated as present in an independent SNP analysis dataset of 19 SCLC tumors obtained from Zhao et al. One of these copy number gains, on 18q, was correlated with sensitivity of SCLC cell lines to the targeted cancer drug ABT-737. The clinical relevance of the 18q21 gain was then verified by FISH analysis of SCLC tumors. The genes residing in the 18q21 marker region were shown to be overexpressed in the sensitive cell lines.

Materials and Methods

Cell culture.

The following SCLC cell lines were obtained from ATCC (Manassis, Va.): NCI-H889, NCI-H1963, NCI-H1417, NCI-H146, NCI-H187, DMS53, NCI-H510, NCI-H1209, NCI-H526, NCI-H211, NCI-H345, NCI-H524, NCI-H69, NCI-H748, DMS79, NCI-H711, SHP77, NCI-446, NCI-H1048, NCI-H82, NCI-H196, SW1271, H69AR. All cells were cultured in the ATCC recommended media at 37° C. in a humidified atmosphere containing 5% CO₂. Genomic DNA was isolated from the cell lines using a DNAeasy kit (Qiagen, Valencia, Calif.). Comparative Genomic Hybridization.

Genomic DNA from the SCLC cell lines was run on 100K SNP genotyping array sets (Affymetrix, Santa Clara, Calif.). Each 100K set consists of two 50K arrays, HindIII and XbaI. Briefly, 250 ng of genomic DNA from each cell line was digested with the corresponding restriction enzyme (HindIII or XbaI, New England Biolabs, Boston, Mass.). Adapters were ligated to the digested DNA, followed by PCR amplification with Pfx DNA polymerase (Invitrogen, Carlsbad, Calif.). The PCR products were purified, fragmented, labeled, and hybridized to the SNP microarray according to the manufacturer's protocol. After a 16-hour hybridization, the arrays were scanned, and the data were processed using the Affymetrix GTYPE software to create copy number (.cnt) files containing information on the inferred copy number for each probeset (SNP). The GTYPE software generates an inferred copy number for each SNP by comparing the signal intensity for the sample with an internal data set from a healthy population, which is included in the GTYPE software. The .cnt files contained combined information from both arrays in the set. These files were converted into .txt files and loaded into an internally developed software program for further analysis.

Applicants' program was used for the graphical display and analysis of multiple .txt files. The data were displayed chromosome by chromosome as a histogram of copy number versus SNP's ordered sequentially along the chromosome. For each SNP, the predicted cytogenetic band as well as any genes between this and the next adjacent SNP were reported. The gene coordinates and cytogenetic band positions were inferred from the Build 35 of the Human Genome. From a selected region of the histogram, for example, 18q21, a summary file can be produced that contains the coordinates of all probesets on the microarray for that region (individual SNP's) with the corresponding copy numbers, cytogenetic bands, gene IDs, names, and the coordinates of all the genes residing in the region (regardless of whether a gene is actually represented by SNP's on the array). In the analysis, contiguous SNP's with a small p-value (p-value <0.08) were considered to be one region.

To facilitate identification of recurrent aberrations, the frequency of copy number change was calculated and plotted for each probeset (SNP) on the microarray, using a threshold of ≧2.8 copies for copy number gains and of ≦1.5 copies for copy number losses. The cell lines were then classified as sensitive and resistant to ABT-737. Fisher's Exact Test was used to identify aberrations in the copy number data that were associated with the sensitivity of cell lines to the Bcl-2 inhibitor. For each SNP, a 2×2 contingency table was constructed for testing the significance of an increase or decrease in copy number in the two groups.

Applicants also obtained from the authors of Zhao et al. study of SCLC, a copy of their raw microarray hybridization data produced in the study reported on in Zhao et al. Applicants analyzed the Zhao et al. raw data for copy number aberrations, and compared the copy number changes identified by Applicants as present in the Zhao data to those identified in Applicants' study of the SCLC cell lines.

Real-Time Quantitative PCR (qPCR).

Primers were designed using the Vector NTI software (Invitrogen) and tested to ensure amplification of single discrete bands with no primer dimers. All primers were synthesized by IDT (Coraville, Iowa). Two independent forward and reverse primer pairs were used for each of the six loci within the 18q21-q22 discriminant region. The primer sequences used are listed in pairs with each pair's approximate location from the 18p terminus, with the forward primers having odd Sequence Identification Numbers (SEQ ID NO's) and the reverse primers having even SEQ ID NO's, and were:

| | | |
|---|---|---|
| From 18p | Sequence | SEQ ID NO |

| | | |
|---|---|---|
| 48MB | TCCTGAGGGTCTTCTCTGTGGAGG | (SEQ ID NO: 1) |
| 48MB | TGTGCCTGGAATACATCTCCGAGA | (SEQ ID NO: 2) |
| 48MB | TAAGACAGATCACCTTCCAAGAGAGACAC | (SEQ ID NO: 3) |
| 48MB | CACAGGCTGCACTTTAGAGGCAA | (SEQ ID NO: 4) |
| 53MB | CAACAGCATGTGCTTCATAGTTGCC | (SEQ ID NO: 5) |
| 53MB | CGACAGCACTGCCCACTCTAGTAATAG | (SEQ ID NO: 6) |
| 53MB | AACAAACACTTGAAGACACTGAAGAACAAC | (SEQ ID NO: 7) |
| 53MB | TGCTCTCAACTGAAAATGGCTATATGTC | (SEQ ID NO: 8) |
| 54MB | TCTTCCAGGGCACCTTACTGTCC | (SEQ ID NO: 9) |
| 54MB | ACCAGCAACCCCATTCCGAG | (SEQ ID NO: 10) |
| 54MB | TTGATGTGTCCCCTGTGCCTTTA | (SEQ ID NO: 11) |
| 54MB | ACAAGTTTTTGCCTCTAGATGACACTGTT | (SEQ ID NO: 12) |
| 55MB | AACCCGAGGAAGTCTAAATGAATAAT | (SEQ ID NO: 13) |
| 55MB | CACACCCAGTTACCCCTGTTATTAAC | (SEQ ID NO: 14) |
| 55MB | TCCTCTCTCATCTGTAGTCTGGCTTTA | (SEQ ID NO: 15) |
| 55MB | AAACTATAATAGCAATCTGTGCCCAA | (SEQ ID NO: 16) |
| 59MB | AGCATTGGTGCGTGTGGTGC | (SEQ ID NO: 17) |
| 59MB | CCTCTTGGTGGAATCTAGGATCAGG | (SEQ ID NO: 18) |
| 59MB | TTCAAGTGAAGTTACCTAATGCTCCC | (SEQ ID NO: 19) |
| 59MB | CCTGGGGTACAGAAATACTTAGTGAT | (SEQ ID NO: 20) |
| 62MB | TTGGAAAGTCTGGATGGGAATCTTTT | (SEQ ID NO: 21) |
| 62MB | AGGGGATTTAACCTACCTTTGTTTC | (SEQ ID NO: 22) |
| 62MB | ATGACAATTAAATTATCACGCTTCCA | (SEQ ID NO: 23) |
| 62MB | TTCTTCTTGTCAGCAGCCACTTATCA | (SEQ ID NO: 24) |

Real-time, quantitative PCR was conducted on an iCycler thermocycler (Bio-Rad, Hercules, Calif.) using SYBR Green qPCR supermix UDG (Invitrogen). Each reaction was run in triplicate and contained 10 ng of purified genomic DNA along with 300 nM of each primer in a final volume of 50 μl. The cycling parameters used were: 95° C. for 3 min.; 35 cycles of 95° C. for 10 sec.; 57° C. for 45 sec. Melting curves were performed to ensure that only a single amplicon was produced and samples were run on a 4% agarose gel (Invitrogen) to confirm specificity. Data analysis was performed in the linear regression software DART-PCR v1.0, see Peirson, S. N., et al., “Experimental validation of novel and conventional approaches to quantitative real-time PCR data analysis”, Nucleic Acids Res., 31: e73, 2003, using raw thermocycler values. Normalization of sample input was conducted using geometric averaging software GeNorm v3.3 (23) to GAPDH, β-2 microglobulin, YWHAZ, RPL13a, and PLP-1, see Vandesompele, J, De Preter K et. al., “Accurate normalization of real-time quantitative RT-PCR data by geometric averaging of multiple internal control genes”, Genome Biol., 2002 Jun. 18; 3 (7):RESEARCH0034, Epub 2002 Jun. 18, PMID 12184808 [PubMed—indexed for MEDLINE]. The copy number for each locus evaluated was determined by establishing the normalized qPCR output for the sample and dividing this value by the normalized qPCR output of a control genomic DNA (Clontech, Mountain View, Calif.) and multiplying this value by two. Each qPCR copy number estimate is the average value for two independent primer sets (mean CV 11.5%).

### Fluorescent In Situ Hybridization.

A tissue microarray containing primary SCLC tumors from 62 patients provided by Dr. Guido Sauter of the Department of Pathology, University Medical Center, Hamburg-Eppendorf, was analyzed by FISH using a commercially available dual-color FISH probe targeting 18q21 (LSI Bcl-2 Break-apart probe, Abbott Molecular). This LSI Bcl-2 FISH probe contains two probes labeled in different fluorescent colors that hybridize adjacent to each side of the Bcl-2 locus at 18q21.3, but does not hybridize to any of the genomic sequence of Bcl-2. The slides were deparaffinized for 10 minutes in Xylol, rinsed in 95% EtOH, air-dried, incubated in a Pretreatment Solution (Abbott Molecular) for 15 minutes at 80° C., rinsed in water, incubated in a Protease Buffer (Abbott Molecular) for 2.5 to 5 hours, rinsed in water, dehydrated for 3 min each in 70, 80, and 95% EtOH, and air-dried. 10 μl of the probe mix was applied onto the slide, and the slide was covered, sealed, heated to 72° C. for 5 minutes, and hybridized overnight at 37° C. in a wet chamber. The slides were then washed with a wash buffer containing 2×SSC and 0.3% NP40 (pH 7-7.5) for 2 minutes at 75° C., rinsed in water at room temperature, air-dried, mounted with a DAPI solution and a 24×50 mm coverslip, and examined under an epifluorescence microscope. For each tissue sample, the range of red and green FISH signals corresponding to the Bcl-2 locus was recorded. An average copy number per spot was then calculated based on the minimal and maximal number of FISH signals per cell nucleus in each tissue spot. Copy number groups were then built according to the following criteria:
(1) 1-2 signals=average copy number <2.5;
(2) 3-4 signals=average copy number <4.5;
(3) 5-6 signals=average copy number <6.5; and
(4) 7-10 signals=average copy number >6.5.

### Microarray Analysis of Gene Expression.

Total RNA was isolated by using the Trizol reagent (Invitrogen,) and purified on RNeasy columns (Qiagen, Valencia, Calif.). Labeled cRNA was prepared according to the microarray manufacturer's protocol and hybridized to human U133A 2.0 arrays (Affymetrix, Santa Clara, Calif.). The U133A 2.0 chips contain 14,500 well-characterized genes, as well as several thousand ESTs. The microarray data files were loaded into the Rosetta Resolver™ software for analysis and the intensity values for all probesets were normalized using the Resolver's Experimental Definition. The intensity values for the probesets corresponding to genes within the amplified regions were normalized across each gene and compared in heatmaps using the Spotfire™ software.

### Results

Table 1 summarizes all copy number abnormalities that Applicants identified as (i) present in ≧40% of the tested cell lines, and (ii) present in ≧40% of the 19 SCLC tumors from the dataset of Zhao et al., and (iii) as not previously reported in the literature, including not reported by Zhao et al. The list of identified novel aberrations includes gains of 2q, 6p, 7p, 9q, 11p, 11q, 12p, 12q, 13q, 14q, 17q, 18q, 20p, 20q, 21q, and 22q and losses of 110q21.1. All of these were confirmed by real-time qPCR in selected cell lines. As can be seen in Table 1, all of these identified novel aberrations are relatively short (about 70 kb to about 3.6 Mb). The mean spacing between the SNPs on the 100K SNP array used in this study is 23.6 kb, thus permitting identification of very short regions of gains and losses. It is possible that some of the newly detected recurrent copy number changes represent copy number polymorphisms, as opposed to disease driven changes. However, this is only a remote possibility, because the copy number was determined relative to a panel of 110 normal individuals, see Huang, J., et al., “Whole genome DNA copy number changes identified by high density oligonucleotide arrays”, Hum. Genomics, 1: 287-299, 2004.

TABLE 1

| | | | | | Genes in this |
|---|---|---|---|---|---|
| | | | | | locus with |
| | | | | | reported |
| Copy Number | | | Frequency | | association with |
| Abnormality | Length | | in cell lines | Frequency in tumors | cancer |
| Gain of | 420 | kb | 61% | 66% | |
| 2q37.1-q37.2 | | | | | |
| Gain of | 3.63 | Mb | 69% | 63% | CK2B, MSH5 |
| 6p21.31 | | | | | |
| Gain of 7p22.1 | 270 | kb | 69% | 54% | RAC1 |
| Gain of 7p14.3 | 40 | kb | 75% | 41% | |
| Gain of | 560 | kb | 47% | 42% | |
| 7q11.21 | | | | | |
| Gain of 7q22.1 | 2.51 | Mb | 71% | 60% | RFC2, FZD9, |
| | | | | | BCL7B |
| Gain of 7q36 | 190 | kb | 55% | 80% | PTPRN2 |
| Gain of 9q34.1 | 130 | kb | 72% | 54% | ABL1 |
| Gain of 9q34.2 | 1.86 | Mb | 58% | 63% | |
| Loss of | 480 | kb | 85% | 98% | |
| 10q21.1 | | | | | |
| Loss of | 340 | kb | 53% | 42% | |
| 10q21.1 | | | | | |
| Loss of | 189 | Mb | 57% | 44% | |
| 11p11.12 | | | | | |
| Gain of | 230 | kb | 41% | 46% | |
| 11q13.2-q13.3 | | | | | |
| Gain of | 390 | kb | 59% | 60% | |
| 11q13.4 | | | | | |
| Gain of | 390 | kb | 88% | 81% | |
| 11q23.3 | | | | | |
| Gain of 12p13 | 430 | kb | 74% | 41% | DDX6, BCL9L, |
| | | | | | FOXR1, TMEM24 |
| Gain of | 48 | kb | 52% | 96% | |
| 12p13.31 | | | | | |
| Gain of | 490 | kb | 57% | 83% | TNFRSF1A, |
| 12q13.12 | | | | | CHD4 |
| Gain of | 340 | kb | 73% | 58% | BAX inhibitor-1, |
| 12q14.2 | | | | | FAIM-2 |
| Gain of | 98 | kb | 65% | 70% | RASSF3 |
| 12q24.11 | | | | | |
| Gain of | 260 | kb | 80% | 67% | |
| 12q24.12 | | | | | |
| Gain of | 180 | kb | 86% | 46% | |
| 12q24.13 | | | | | |
| Gain of | 10 | kb | 61% | 58% | |
| 12q24.33 | | | | | |
| Gain of 13q34 | 750 | kb | 55% | 85% | MMP17 |
| Gain of 14q11 | 130 | kb | 43% | 47% | |
| Gain of | 70 | kb | 48% | 40% | ER2 |
| 14q23.2 | | | | | |
| Gain of | 410 | kb | 46% | 45% | |
| 14q24.3 | | | | | |
| Gain of | 1.05 | Mb | 54% | 47% | |
| 14q24.3 | | | | | |
| Gain of | 160 | kb | 51% | 52% | CHES1 |
| 14q24.3-q31 | | | | | |
| Gain of | 2.36 | Mb | 50% | 56% | |
| 14q32.12 | | | | | |
| Gain of | 6 | Mb | 48% | 61% | TCL6 |
| 14q32.1-32.2 | | | | | |
| Gain of | 1.84 | Mb | 83% | 78% | TMEM121 |
| 14q32.33 | | | | | |
| Gain of | 230 | kb | 43% | 70% | |
| 17q21.33 | | | | | |
| Gain of | 2.62 | Mb | 53% | 77% | |
| 17q24.3-q25.1 | | | | | |
| Gain of | 1.12 | Mb | 59% | 61% | |
| 17q25.3 | | | | | |
| Gain of 18q12 | 190 | kb | 46% | 54% | |
| Gain of | 370 | kb | 48% | 51% | |
| 18q21.1 | | | | | |
| Gain of 18q22-q23 | 400 | kb | 46% | 88% | |
| Gain of 20p13 | 370 | kb | 57% | 45% | |
| Gain of 20p13-p12 | 190 | kb | 59% | 49% | |
| Gain of | 300 | kb | 62% | 41% | |
| 20p11.23 | | | | | |
| Gain of | 790 | kb | 52% | 40% | |
| 20p11.21 | | | | | |
| Gain of | 230 | kb | 64% | 98% | |
| 20q11.21 | | | | | |
| Gain of | 280 | kb | 35% | 56% | |
| 20q11.23 | | | | | |
| Gain of 20q12-q13.1 | 190 | kb | 43% | 98% | |
| Gain of | 2.45 | Mb | 60% | 58% | PREX1, CSE1L |
| 20q13.1-q13.13 | | | | | |
| Gain of | 40 | kb | 42% | 84% | RAB22A |
| 20q13.32-13.33 | | | | | |
| Gain of | 2.74 | Mb | 47% | 57% | |
| 20q13.3 | | | | | |
| Gain of | 1.47 | Mb | 57% | 69% | |
| 21q22.3 | | | | | |
| Gain of | 66 | kb | 65% | 61% | |
| 22q13.1 | | | | | |

The 23 SCLC cell lines were tested for sensitivity to ABT-737 using the procedure described in Oltersdorf, T., “An inhibitor of Bcl-2 family proteins induces regression of solid tumours”, Nature, 435: 677-681, 2005, with a cell line classified as sensitive if its EC50 <1 μM and as resistant if its EC50 >10 μM. The sensitive cell line group consisted of NCI-H889, NCI-H1963, NCI-H1417, NCI-H146, NCI-H187, DMS 53, NCI-H510, NCI-H209, NCI-H526, NCI-H211, NCI-H345, and NCI-H524 and the resistant cell line group was comprised of NCI-H82, NCI-H196, SW1271, and H69AR.

To identify potential genomic correlates of the sensitivity of SCLC cells to ABT-737, we developed a bioinformatics approach that identifies regions of chromosomal aberrations that discriminate between the sensitive and resistant groups. Our program tested for statistical significance using Fisher's Exact Test to determine if a SNP shows preferential gain/loss in the sensitive or resistant group. The copy number thresholds for amplifications and deletions were set at 2.8 and 1.5, respectively. Contiguous regions of probesets (SNPs) with low table and two-sided p-values were subjected to further analysis. The top discriminating aberration represents a long region of chromosome 18, starting at nucleotide position 45704096 and ending at nucleotide position 74199087 and spanning the chromosomal bands 18q21.1 through 18q22.1 (nucleotide positions are from Build 35 of the Human Genome Map).

Real-time qPCR was then applied to validate the 18q21 region identified in the copy number analysis as a potential stratification marker. Two different primer sets run in triplicate were used to evaluate six loci starting at 48 Mb from the chromosome 18p terminus (18q21.1) and ending at 62 Mb from the chromosome 18p terminus (18q22). The qPCR results are shown in FIG. 1, with the copy number measured at each locus plotted against sensitivity to ABT-737. FIG. 1 shows segregation between the sensitive and resistant lines based on the copy number of the test locus (ANOVA test p-value <0.0001), thus confirming the copy number analysis. The sensitive lines carry an amplification of the region under consideration (3 to 7 copies), whereas the resistant lines display a normal copy number. Further, the most sensitive lines (H889, H1963, H1417, and H146) have the highest Bcl-2 copy number (4 or 5 copies).

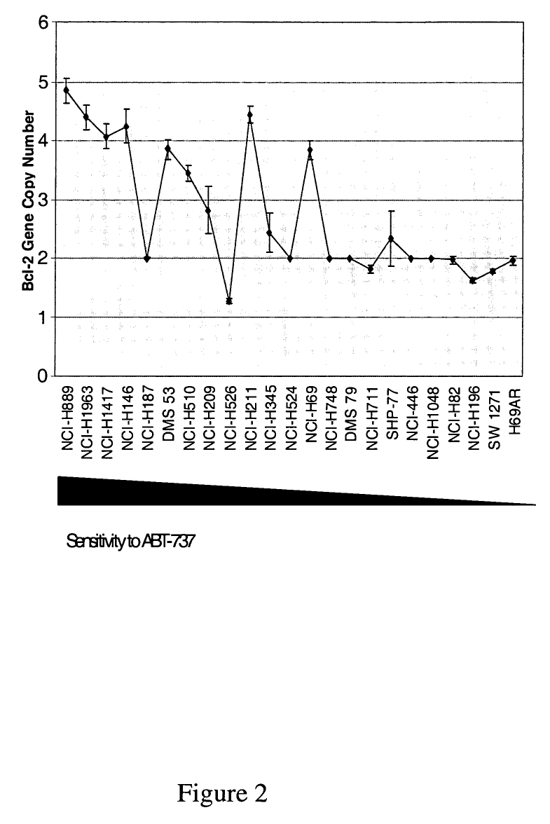

Notably, the Bcl-2 gene (p-value 0.04), the target of ABT-737, is located within the 18q21-q22 discriminant region at 18q21.3, which led to investigation of whether the sensitivity of a cell line to the drug may be determined by the amplification status of the Bcl-2 gene. FIG. 2 illustrates the relationship between the Bcl-2 gene copy number and the sensitivity of the SCLC cell lines. The cell lines are arranged from left to right in the order of decreasing sensitivity to the drug, as determined by the EC₅₀ values for the cell lines from Oltersdorf, T., et al., “An inhibitor of Bcl-2 family proteins induces regression of solid tumours”, Nature, 435: 677-681, 2005.

The copy number for each cell line in FIG. 2 is the average of the copy numbers for 17 SNP's within the Bcl-2 gene measured by the 100K mapping array set. The copy number for the NOXA and Bcl-w genes was the number determined for at least three continguous SNP's surrounding their gene loci. It is clear from the plot that the sensitivity of the SCLC cell lines correlates with the Bcl-2 copy number. The most sensitive lines (H889, H1963, H1417, and H146) have the highest Bcl-2 copy number (4 or 5 copies). Another apoptosis-related gene (NOXA), whose product promotes degradation of Mcl-1, is located next to Bcl-2 and has a similar copy number profile. There are two outliers in this dataset, which are sensitive, but have a normal copy number of the Bcl-2 gene (H187 and H526). However, both H187 and H526 cell lines have copy number gain of the Bcl-w gene at 14q11.2, which is also a target of the drug. Their sensitivity to ABT-737 is attributed to the extra copy of the Bcl-w gene at 14q11.2. A similar plot did not show any correlation of sensitivity to Bcl-XL copy number gain, although copy number gain was seen in some cell lines. Thus, we established a correlation between the amplification of Bcl-2 and NOXA on 18q21.3 and the sensitivity of SCLC cell lines to ABT-737. This observation is consistent with the mechanism of action of the drug and suggests that the single-agent sensitivity of a cell line to the drug may be determined by the copy number status of 18q21, particularly the 18q21.3 locus of Bcl-2 and NOXA.

The relative expression of the 18q genes in the ABT-737 sensitive and resistant SCLC cell lines was profiled with expression microarrays as described above. The 12 most sensitive cell lines and four resistant lines were analyzed for expression of all genes located on the discriminant region on 18q21-q22 and present on the Affymetrix U133A microarray used. The genes in the amplified region were found overexpressed in the sensitive lines relative to the resistant ones. Overall, the finding of overexpression of the 18q21-q22 genes implies a significant degree of correlation between gene amplification and gene overexpression. These data further support for the selection of the 18q21-q22 copy number gain as a patient stratification biomarker in SCLC.

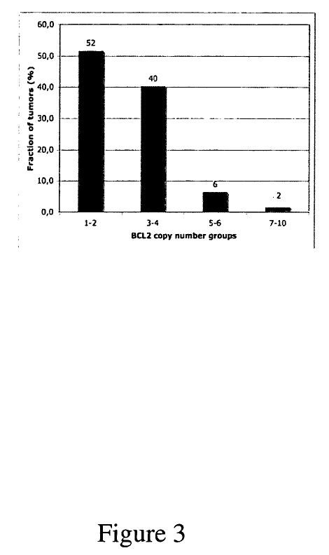

To determine the clinical relevance of the 18q21-q22 marker, the Bcl-2 copy number in SCLC tumors using FISH with a commercially available Bcl-2 locus probe set. Although the commercial FISH′ probe used did not contain any of the Bcl-2 gene sequence itself, the probe used contain sequences that hybridize on both sides of the gene, and a continguous copy number increase seen with both parts of this probe is believed by Applicants to include a gain of the Bcl-2 locus also. Applicants' analysis included SCLC tumors from 62 patients arrayed on a tissue microarray. The data is shown in FIG. 3. Copy number gains were seen in 48% of the cohort, with low-level amplifications of the Bcl-2 gene present in 40% of the patients (25 out of 62) and high-level amplifications in 8% of the tumors (5 out of 62). This finding is consistent with the copy number data from the SCLC cell lines, as most copy number changes in the cell lines were also low-level gains. The percentage of lines carrying the aberration was also similar (40%).

### Example 2

The following Example 2 describes determination of levels of pro-GRP in four cell lines showing elevated copy number for the Bcl-2 locus. The cell lines tested were NCI-H889, NCI-H146, DMS53 and NCI-H510, and these cell lines had shown sensitivity to the Bcl-2 inhibitor. The cells from each were cultured for seven days at 37 degrees C., then the medium was collected and stored at −70 degrees C. for one week. The medium from each cell line was thawed on ice, and then tested by a commercially available ELISA assay (distributed by IBL and made by Advanced Life Sciences Institute, Japan) for pro-GRP levels. The pro-GRP levels were estimated for the DMS53 cell line because the OD was outside the top range of the standard curve for the assay. The pro-GRP levels in picograms pro-GRP per milliliter per micrograms of total protein (pg pro-GRP/ml/μg protein) were:

| | |
|---|---|
| NCI-H889 | about 2.9 |
| NCI-H146 | about 0.1 |
| DMS53 | about 9.5 |
| NCI-H510 | about 2.0. |

Higher levels of pro-GRP correlating to the presence of the chromosomal copy number increase were seen in the NCI-H889, DMS53 and NCI-510 cell lines.

```
<200> SEQUENCE CHARACTERISTICS:
<210> SEQ ID NO: 1
<211> LENGTH: 24
<212> TYPE: DNA
<213> ORGANISM: Artificial
<220> FEATURE:
    <221> NAME/KEY: Misc_feature
    <222> LOCATION: 1-24
    <223> OTHER INFORMATION: sequence is synthesized
    <400> SEQUENCE: 1
TCCTGAGGGT CTTCTCTGTG GAGG
<200> SEQUENCE CHARACTERISTICS:
<210> SEQ ID NO: 2
<211> LENGTH: 24
<212> TYPE: DNA
<213> ORGANISM: Artificial
<220> FEATURE:
    <221> NAME/KEY: Misc_feature
    <222> LOCATION: 1-24
    <223> OTHER INFORMATION: sequence is synthesized
    <400> SEQUENCE: 2
TGTGCCTGGA ATACATCTCC GAGA
<200> SEQUENCE CHARACTERISTICS:
<210> SEQ ID NO: 3
<211> LENGTH: 29
<212> TYPE: DNA
<213> ORGANISM: Artificial
<220> FEATURE:
    <221> NAME/KEY: Misc_feature
    <222> LOCATION: 1-29
    <223> OTHER INFORMATION: sequence is synthesized
    <400> SEQUENCE: 3
TAAGACAGAT CACCTTCCAA GAGAGACAC
<200> SEQUENCE CHARACTERISTICS:
<210> SEQ ID NO: 4
<211> LENGTH: 23
<212> TYPE: DNA
<213> ORGANISM: Artificial
<220> FEATURE:
    <221> NAME/KEY: Misc_feature
    <222> LOCATION: 1-23
    <223> OTHER INFORMATION: sequence is synthesized
    <400> SEQUENCE: 4
CACAGGCTGC ACTTTAGAGG CAA
<200> SEQUENCE CHARACTERISTICS:
<210> SEQ ID NO: 5
<211> LENGTH: 25
<212> TYPE: DNA
<213> ORGANISM: Artificial
<220> FEATURE:
    <221> NAME/KEY: Misc_feature
    <222> LOCATION: 1-25
    <223> OTHER INFORMATION: sequence is synthesized
    <400> SEQUENCE: 5
CAACAGCATG TGCTTCATAG TTGCC
<200> SEQUENCE CHARACTERISTICS:
<210> SEQ ID NO: 6
<211> LENGTH: 29
<212> TYPE: DNA
<213> ORGANISM: Artificial
<220> FEATURE:
    <221> NAME/KEY: Misc_feature
    <222> LOCATION: 1-29
    <223> OTHER INEORMATION: sequence is synthesized
    <400> SEQUENCE: 6
CGACAGCACT GCCCACTC TAGTAATAG
<200> SEQUENCE CHARACTERISTICS:
<210> SEQ ID NO: 7
<211> LENGTH: 39
<212> TYPE: DNA
<213> ORGANISM: Artificial
<220> FEATURE:
    <221> NAME/KEY: Misc_feature
    <222> LOCATION: 1-30
    <223> OTHER INFORMATION: sequence is synthesized
    <400> SEQUENCE: 7
AACAAACACT TGAAGACACT GAAGAACAAC
<200> SEQUENCE CHARACTERISTICS:
<210> SEQ ID NO: 8
<211> LENGTH: 28
<212> TYPE: DNA
<213> ORGANISM: Artificial
<220> FEATURE:
    <221> NAME/KEY: Misc_feature
    <222> LOCATION: 1-28
    <223> OTHER INFORMATION: sequence is synthesized
    <400> SEQUENCE: 8
TGCTCTCAAC TGAAAATGGC TATATGTC
<200> SEQUENCE CHARACTERISTICS:
<210> SEQ ID NO: 9
<211> LENGTH: 23
<212> TYPE: DNA
<213> ORGANISM: Artificial
<220> FEATURE:
    <221> NAME/KEY: Misc_feature
    <222> LOCATION: 1-23
    <223> OTHER INFORMATION: sequence is synthesized
    <400> SEQUENCE: 9
TCTTCCAGGG CACCTTACTG TCC
<200> SEQUENCE CHARACTERISTICS:
<210> SEQ ID NO: 10
<211> LENGTH: 20
<212> TYPE: DNA
<213> ORGANISM: Artificial
<220> FEATURE:
    <221> NAME/KEY: Misc_feature
    <222> LOCATION: 1-20
    <223> OTHER INFORMATION: sequence is synthesized
    <400> SEQUENCE: 10
ACCAGCAACC CCATTCCGAG
<200> SEQUENCE CHARACTERISTICS:
<210> SEQ ID NO: 11
<211> LENGTH: 23
<212> TYPE: DNA
<213> ORGANISM: Artificial
<220> FEATURE:
    <221> NAME/KEY: Misc_feature
    <222> LOCATION: 1-23
    <223> OTHER INFORMATION: sequence is synthesized
    <400> SEQUENCE: 11
TTGATGTGTC CCCTGTGCCT TTA
<200> SEQUENCE CHARACTERISTICS:
<210> SEQ ID NO: 12
<211> LENGTH: 29
<212> TYPE: DNA
<213> ORGANISM: Artificial
<220> FEATURE:
    <221> NAME/KEY: Misc_feature
    <222> LOCATION: 1-29
    <223> OTHER INFORMATION: sequence is synthesized
    <400> SEQUENCE: 12
ACAAGTTTTT GCCTCTAGAT GACACTGTT
<200> SEQUENCE CHARACTERISTICS:
<210> SEQ ID NO: 13
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 13
AACCCGAGGA AGTCTAAATG AATAAT
<200> SEQUENCE CHARACTERISTICS:
<210> SEQ ID NO: 14
<211> LENGTH: 25
<212> TYPE: DNA
<213> ORGANISM: Artificial
<220> FEATURE:
    <221> NAME/KEY: Misc_feature
    <222> LOCATION: 1-25
    <223> OTHER INFORMATION: sequence is synthesized
    <400> SEQUENCE: 14
CACACCCAGT TACCCCTGTTA TTAAC
<200> SEQUENCE CHARACTERISTICS:
<210> SEQ ID NO: 15
<211> LENGTH: 27
<212> TYPE: DNA
<213> ORGANISM: Artificial
<220> FEATURE:
    <221> NAME/KEY: Misc_feature
    <222> LOCATION: 1-27
    <223> OTHER INFORMATION: sequence is synthesized
    <400> SEQUENCE: 15
TCCTCTCTCA TCTGTAGTCT GGCTTTA
<200> SEQUENCE CHARACTERISTICS:
<210> SEQ ID NO: 16
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 16
AAACTATAAT AGCAATCTGT GCCCAA
<200> SEQUENCE CHARACTERISTICS:
<210> SEQ ID NO: 17
<211> LENGTH: 20
<212> TYPE: DNA
<213> ORGANISM: Artificial
<220> FEATURE:
    <221> NAME/KEY: Misc_feature
    <222> LOCATION: 1-20
    <223> OTHER INFORMATION: sequence is synthesized
    <400> SEQUENCE: 17
AGCATTGGTG CGTGTGGTGC
<200> SEQUENCE CHARACTERISTICS:
<210> SEQ ID NO: 18
<211> LENGTH: 25
<212> TYPE: DNA
<213> ORGANISM: Artificial
<220> FEATURE:
    <221> NAME/KEY: Misc_feature
    <222> LOCATION: 1-25
    <223> OTHER INFORMATION: sequence is synthesized
    <400> SEQUENCE: 18
CCTCTTGGTG GAATCTAGGA TCAGG
<200> SEQUENCE CHARACTERISTICS:
<210> SEQ ID NO: 19
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 19
TTCAAGTGAA GTTACCTAAT GCTCCC
<200> SEQUENCE CHARACTERISTICS:
<210> SEQ ID NO: 20
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 20
CCTGGGGTAC AGAAATACTT AGTGAT
<200> SEQUENCE CHARACTERISTICS:
<210> SEQ ID NO: 21
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER lNFORMATION: sequence is synthesized
    <400> SEQUENCE: 21
TTGGAAAGTC TGGATGGGAA TCTTTT
<200> SEQUENCE CHARACTERISTICS:
<210> SEQ ID NO: 22
<211> LENGTH: 25
<212> TYPE: DNA
<213> ORGANISM: Artificial
<220> FEATURE:
    <221> NAME/KEY: Misc_feature
    <222> LOCATION: 1-25
    <223> OTHER INFORMATION: sequence is synthesized
    <400> SEQUENCE: 22
AGGGGATTTA ACCTACCTTT GTTTC
<200> SEQUENCE CHARACTERISTICS:
<210> SEQ ID NO: 23
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 23
ATGACAATTA AATTATCACG CTTCCA
<200> SEQUENCE CHARACTERISTICS:
<210> SEQ ID NO: 24
<211> LENGTH: 26
<212> TYPE: DNA
<213> ORGANISM: Artificial
<220> FEATURE:
    <221> NAME/KEY: Misc_feature
    <222> LOCATION: 1-26
    <223> OTHER INFORMATION: sequence is synthesized
    <400> SEQUENCE: 24
TTCTTCTTGT CAGCAGCCAC TTATCA
```

What is claimed is:

1. A method for identifying a patient with cancer as eligible to receive Bcl-2-family inhibitor therapy comprising:
(a) providing a tissue sample from a patient;
(b) determining presence or absence of increased levels of at least one of (i) an expressed protein of pro-GRP, (ii) an expressed protein of a pro-GRP precursor, or (iii) fragments thereof; and
(c) classifying the patient as eligible to receive Bcl-family inhibitor therapy where the tissue sample is determined as having increased levels of at least one of (i) an expressed protein of pro-GRP, (ii) an expressed protein of a pro-GRP precursor, or (iii) fragments thereof.

2. The method of claim 1, wherein the tissue sample comprises a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or plasma fraction of a blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample a fresh frozen tissue sample or a paraffin embedded tissue sample.

3. The method of claim 1, wherein the tissue sample is from a patient with a cancer selected from the group consisting of small cell lung carcinoma and a lymphoma.

4. The method of claim 1, wherein the patient is classified as eligible to receive N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide or analogs thereof.

5. The method of claim 1, wherein the patient is classified as eligible to receive an anti-sense therapy compound designed to bind to Bcl-2.

6. The method of claim 1, wherein the determining step (b) is performed by immunoassay to a peripheral blood sample or plasma or serum fraction thereof.

7. The method of claim 6, wherein the immunoassay is a sandwich immunoassay.

8. The method of claim 6, wherein the immunoassay is an ELISA.

9. The method of claim 6, wherein the determining step (b) is performed on an automated immunoassay instrument.

10. A method for monitoring a patient being treated with anti-Bcl-2-family therapy comprising:
(a) providing a peripheral blood sample from a cancer patient;
(b) determining presence or absence of increased levels in the peripheral blood sample of at least one of (i) an expressed protein of pro-GRP, (ii) an expressed protein of a pro-GRP precursor, or (iii) fragments thereof;
(c) comparing determined levels in the peripheral blood sample of at least one of (i) an expressed protein of pro-GRP, (ii) an expressed protein of a pro-GRP precursor, or (iii) fragments thereof to a baseline level of at least one of (i) an expressed protein of pro-GRP, (ii) an expressed protein of a pro-GRP precursor, or (iii) fragments thereof, wherein the baseline level is determined in a peripheral blood sample from the patient obtained before or at onset of therapy.

11. The method of claim 10 wherein the cancer is selected from the group consisting of small cell lung carcinoma and a lymphoma.

12. The method of claim 10, wherein the levels of at least one of (i) an expressed protein of pro-GRP, (ii) an expressed protein of a pro-GRP precursor, or (iii) fragments thereof are determined by immunoassay.

13. The method of claim 10, wherein the patient is being treated with N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide or analogs thereof.

14. The method of claim 10, wherein the patient is being treated with an anti-sense therapy compound designed to bind to Bcl-2.

15. The method of claim 10, wherein the determining step (b) is performed by a sandwich immunoassay.

16. The method of claim 10, wherein the determining step (b) is performed on an automated immunoassay instrument.

17. The method of claim 13, wherein the patient is being treated with combination therapy.

18. The method of claim 10 further comprising processing the peripheral blood sample to produce a plasma fraction, which is then used in the determining step (b).

19. The method of claim 10, wherein the determining step (b) is performed by an immunoassay using a chemiluminescent label.

20. The method of claim 10, wherein the tissue sample comprises a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or plasma fraction of a peripheral blood sample, a tumor or suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample a fresh frozen tissue sample or a paraffin embedded tissue sample.
